(19) United States
(12) Patent Application Publication
(10) Pub. No.: US 2022/0008246 A1
(43) Pub. Date: Jan. 13, 2022

(54) SYSTEMIC CRYOTHERAPY DEVICE WITH ENGINE ROOM ASSEMBLY

(71) Applicant: CRYO SCIENCE SP. Z O.O., Wroclaw (PL)

(72) Inventors: Grzegorz LEMPART, Wroclaw (PL); Mateusz ZYCH, Wroclaw (PL); Kamil KEDZIERSKI, Wroclaw (PL); Dariusz CZERMINSKI, Wroclaw (PL)

(21) Appl. No.: 17/291,848

(22) PCT Filed: Nov. 6, 2019
(86) PCT No.: PCT/PL2019/050060

§ 371 Date: May 6, 2021

(30) Foreign Application Priority Data
Nov. 6, 2018 (PL) P.427651

(51) Int. Cl. A61F 7/00

(52) U.S. Cl. CPC A61F 7/0053; A61F 2007/0096; A61F 2007/0064

(57) ABSTRACT

The subject of the invention is a device for systemic cryotherapy with an engine room assembly (22) comprising a cryochamber and the engine room assembly (22), which comprises an upper inlet channel housing (8), which is connected, on the bottom, to a ventilation fitting housing (1) connected, on the bottom, to a heat exchanger (2) housing, wherein a cryogenic liquid supply (19) and cryogenic liquid vapour discharge (20) system is located on the side wall, and the lower flange of the heat exchanger (2) is connected to the lower outlet channel housing (3), and the device is connected to a power supply system, characterised in that the upper inlet channel housing (8) is L-shaped, and a duct fan (15) is located in the ventilation fitting housing (1), wherein a heater (9) is located between the ventilation fitting (1) and the heat exchanger (2).

The invention relates to a refrigeration system for a systemic cryotherapy chamber connected to an oxygen concentration control system located inside the chamber. The solution is applied for therapy utilising low temperatures.

From Polish Patent Document PAT.157168B1, a device for performing cryotherapy procedures is known, comprising a chamber for patients and an assembly for feeding air at cryogenic temperature into the chamber, which comprises an air supply circuit with a compressor and a dehumidifier, and an air cooling circuit with a liquid gas container and a heat exchanger. The device comprises a preliminary heat exchanger, a terminal heat exchanger, a spraying element, wherein the preliminary heat exchanger located in the chamber for patients is connected with conduits to the liquid gas container, the heat exchanger and the spraying element, which is also located in the chamber, whereas the terminal heat exchanger is connected with conduits to the heat exchanger and the environment, as well as the air compressor and the dehumidifier. From Patent Application US20150265460, an installation for performing air cryotherapy procedures is known, comprising a closed cryogenic chamber with a heat insulated door and a heat sensor, a refrigeration machine with a condenser and a power supply, and a control unit and a coolant supply line, wherein the refrigeration system has two cascades, and an evaporator with a ventilator is installed into the ceiling or the wall of the cryogenic chamber and is connected by means of the coolant supply line to the double-cascade refrigeration system, wherein a heat control valve is installed at the inlet to the evaporator, and the cryogenic chamber is equipped with a valve for equalizing the pressure with the outside environment. The balancing valve is equipped with an electric heater, similarly to the door frame of the cryogenic chamber. The chamber structure also comprises a control console with a sensor panel on the external surface of the cryogenic chamber. Another Patent Application, US20160089262A1, discloses a cryotherapy apparatus allowing obtaining a uniform temperature on the entire body surface of the patient in the cryotherapy chamber. In use, the apparatus enables active distribution of cold air in a confined space without the undesired consequence of wind shear commonly caused by forced movement of cold air. The refrigeration system comprises a cryogenic liquid container connected with a valve to the cryogenic chamber by means of the liquid supply conduit to the chamber. The chamber comprises a system forcing the vapours of the cryogenic liquid to be blown into the chamber, comprising a motorised fan, an air duct, which is located behind multiple heat exchangers, a deflecting element with a semicircular cross-section, cryogenic liquid vapours as well as a semi-permeable membrane which functions additionally as a shield for the persons inside the cryogenic chamber against contact with the heat exchanger. The technical problem faced by the invention is providing such cryotherapy devices that would be characterised by low use of cryogenic liquids and simultaneously maintaining a constant and low temperature inside the chamber, which would translate into lowering the operating costs of such a device. Surprisingly, the above problems were solved by the present invention.

The subject of the invention is a device for systemic cryotherapy with an engine room assembly comprising a cryochamber and an engine room assembly, which comprises an upper inlet channel housing, which is connected, on the bottom, to a ventilation fitting, housing connected, on the bottom, to a heat exchanger housing, wherein a cryogenic liquid supply and cryogenic liquid vapour discharge system is located on the side wall, and the lower flange of the heat exchanger is connected to the lower outlet channel housing, and the device is connected to the power supply system, characterised in that the upper inlet channel housing is L-shaped, and a duct fan is located in the ventilation fitting housing, wherein a heater is located between the ventilation fitting and the heat exchanger. Wherein the inlet channel housing comprises an air duct inside, its shape corresponding to the shape of the housing, and the longitudinal cross-section of the air duct and the housing is similar to the letter L shape. Inside the lower outlet channel, an internal guide vane is located. It serves to divide and properly distribute the cooled air inside the chamber. Additionally, on the exit from the lower outlet channel, particularly on its damper, a Pt100 temperature sensor in a stainless steel housing is located, responsible for measuring and controlling the temperature in the chamber. In order to place the sensor inside the chamber, a Pt100 mounting sleeve was designed and used, which passes through the walls of the lower outlet channel housing. On the side wall of the inlet channel housing, an oxygen concentration control system inside the cryogenic chamber is located, connected to the cryochamber engine room assembly with a copper pipe, one end of which passes through the wall of the lower outlet channel housing, wherein this end is bended in the direction of the channel axis according the direction of air flow. This provides a spontaneous inflow of air circulating inside the chamber, such that it flows spontaneously (passively) into the copper pipe. An externally insulated heating cable is wound on the copper pipe, and the other end of the copper pipe is connected to the sensor module of the oxygen concentration control system, which module is located in the measuring box. The measuring box is connected directly to the transverse blower, which is mounted to the side wall of the measuring box from the outside. The measuring box is connected to the transverse blower by an opening in the side wall of the measuring box. Additionally, the oxygen concentration control system is connected with a cable to a control and alarm module and to the system and the chamber operation control system. The heat exchanger is fed with liquid nitrogen, which is supplied to the system of pipes and louvers from the pressurised container for liquid gas storage, by means of a cryogenic system equipped with cryogenic electromagnetic valves, and then it cools the air flowing through the exchanger, receiving heat from it during liquid to gas transition called evaporation. Additionally, the quality of heat exchange between the evaporating liquid nitrogen and the circulating air is verified by controlling the temperature of nitrogen vapours leaving the exchanger system, wherein a Pt100 temperature sensor in a ceramic housing is used for this purpose, installed in the Pt100 mounting sleeve, welded into the discharge collector elements and connected to the Pt100 guiding sleeve. The oxygen concentration control system inside the chamber is comprised of a gas detector, the sensor module of which is located in the measuring box, wherein an air sample is drawn from the chamber and supplied by means of a copper pipe equipped with an insulated heating cable, connected on one end to the measuring chamber, an on the other to the lower outlet channel of the chamber, wherein it should be noted that this end is bended in the direction of the channel axis in such a way that the air circulating inside the chamber spontaneously (passively) flows into the copper pipe and into the measuring box. A transverse blower, connected to the measuring box and fixed to one of its walls from the outside, is an additional element providing an appropriate flow of the sampled air. The gas detector is connected to the control and alarm module provided to cooperate with the gas detector and the chamber operation control system. Systemic cryotherapy chamber with an engine room assembly connected to an oxygen concentration control system according to the invention constitutes a device for performing systemic cryotherapy in an atmosphere of cold air. Minimal temperature that can be obtained inside is −140° C. Obtaining such low temperatures requires an efficient and, at the same time, fully safe refrigeration system. For this purpose, a refrigeration system was developed, connected to the oxygen concentration control system, characterised in that the air is drawn from inside the chamber using a fan and next, while flowing through a system of channels and a heat exchanger, it is cooled and fed back to the chamber, from where it is drawn again, wherein the fan is located inside the VENT-HE ventilation fitting connected, on the top side, to the L-shaped upper channel and, on the bottom side, to the heat exchanger, wherein, in the additional space between the exchanger and the ventilation fitting, an electric heater is located, used to heat the air during the chamber drying cycle. Placing the heater between the fan and the heat exchanger significantly impacts the process of heating and drying of the chamber, because the hot air at first heats the heat exchanger, the member of which is the coldest element of the device, and next, at lowered temperature, it dries the interior of the chamber, thus extending the lifetime of the finishing elements by skilfully and slowly heating them. Further, the heat exchanger is connected to the lower outlet channel, in which a guide vane is located, providing a proper air distribution. The air circulation takes place until an adequate treatment temperature is obtained, wherein this temperature is controlled using the Pt100 temperature sensor in a stainless steel housing, located in the damper of the lower channel.

The engine room according to the invention has a compact structure which at the same time simplifies and shortens its assembling process as well as future assembling of the whole device. Mounting the Pt100 temperature sensor in the a stainless steel housing on the exit from the lower outlet channel and using a Teflon mounting sleeve allows, in a simple and, most importantly, precise way, to control the operation of the chamber, and further to assemble it quickly and simply, as well as to disassemble the temperature sensor. Placing the Pt100 temperature sensor in a ceramic housing in a copper mounting sleeve welded into the discharge system collector provides protection for the sensor, in an appropriate way, against mechanical damage and provides adequate contact with the cooling medium, which enables proper control of the heat exchange quality in the exchanger. Placing the vane inside the lower outlet channel makes it possible to maintain a laminar air flow and to simultaneously maintain its higher velocity during the procedure, which allows maintaining a high heat exchange efficiency and provides a proper distribution of cold air inside the cryogenic chamber. Method and placements of mounting of the sensors, as well as utilising a vane diving the cold air stream has significant impact on liquid nitrogen consumption, because it allows maximum utilisation of the liquid nitrogen cooling potential, which makes it possible to reduce its use and provides a high power efficiency of the device. Another advantage of the solution according to the invention is mounting the oxygen concentration control system on the side wall, thus providing easy access to it, which is essential during any maintenance operations, such as e.g. periodic calibrations or sensor replacements. Another advantage of the structure according to the invention is the design of the oxygen concentration control system, which, by utilising a copper pipe, is provided with tightness and appropriate mechanical resistance, and an additional transverse blower connected to the system draws out the sampled air and ensures its proper flow. Further, it should be noted that the copper pipe is equipped with a heating cable and its end, introduced inside the chamber through the lower channel, is bended in the direction of the channel axis according to the direction of airflow. An advantage of such a solution is the simplicity of the structure, which in case of possible malfunction of the blower, due to its design, can ensure constant sampling of air at an appropriate temperature in working range of the detector and prevents mechanical clogging of the pipe, which therefore guarantees reliability of operation of the oxygen concentration control system, which is one of the most crucial security systems of the device.

The embodiments of the subject of the invention are shown on the drawings, on which:

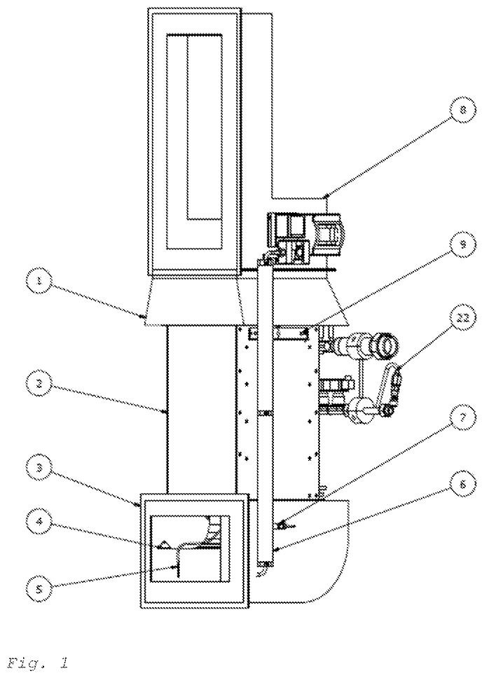

FIG. 1 is a view of the engine room,

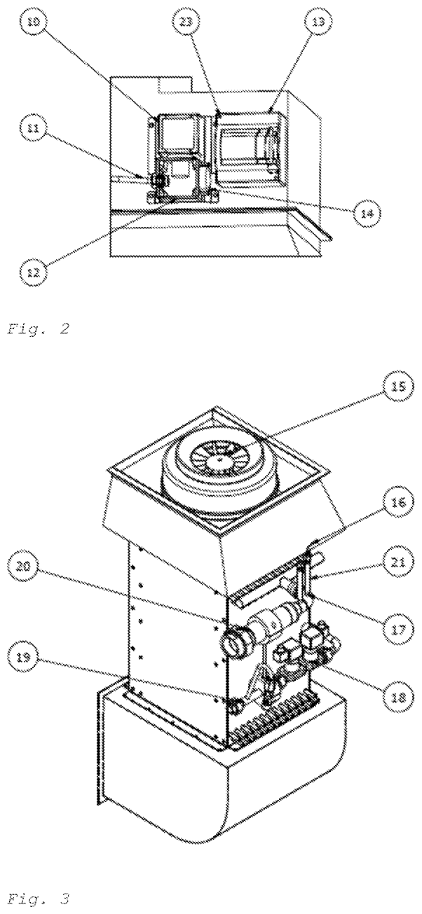

FIG. 2 is a partial cross-sectional view of the oxygen concentration control system,

FIG. 3 presents elements of the cryogenic system, as well as assembling and location of the fan,

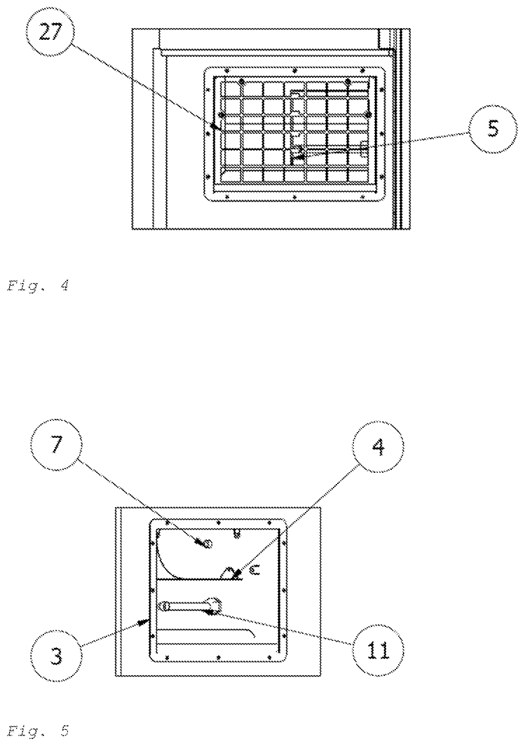

FIG. 4 is an isometric view of the engine room showing the shape of the housing, FIG. 4 presents a method of placing the Pt100 on the damper (in a cross-section),

FIG. 5 presents a method of bending of the measuring pipe,

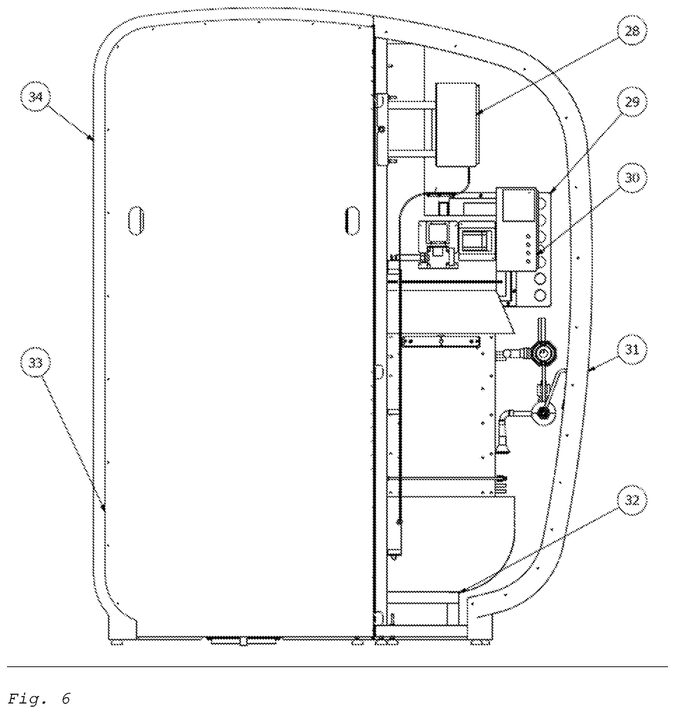

FIG. 6 presents a cryochamber with the engine room system, wherein: 27—a damper of the lower outlet channel, 29—a metal sheet supporting the main monitor, 30—a lower fuse box, 31—an engine room module laminate, 32—a support structure of the engine room, 33—shielding of the chamber module laminate, 34—a chamber module laminate.

### EXAMPLE 1

### Engine Room System Design

The cryochamber engine room 22 is comprised of the upper channel L 8, connected, on the bottom, to the VENT-HE ventilation fitting 1, inside which a duct fan 15 is located, forcing the circulation of cold air flow. The lower part of the ventilation fitting is connected to the heat exchanger 2, wherein, in the space between the fitting and the exchanger pack, a heater 9 is located, which is mounted in a specially prepared mounting opening in the housing of the exchanger. The lower flange of the heat exchanger is connected to the lower outlet channel 3, in which a guide vane 4 is located, for the purpose of properly dividing and distributing the cooled air inside the chamber. Additionally, on the exit from the lower outlet channel, particularly on its damper, a Pt100 temperature sensor in a stainless steel housing 5 is located, responsible for measuring and controlling the temperature in the chamber, wherein, in order to place the sensor inside the chamber, a Pt100 mounting sleeve 7 was designed and used, which passes through the channel walls from the right side. In order to provide liquid nitrogen to the exchanger, a cryogenic power supply system 19 was designed and installed in the heat exchanger, the main elements of which are cryogenic electrovalves 18. Nitrogen in gaseous state is discharged through the discharge system 20, into which a Pt100 mounting sleeve 21 has been welded, connected to the Pt100 guiding sleeve 17 and ending with a cable gland. In this sleeve, a Pt100 temperature sensor is mounted in a ceramic housing 16, measuring the temperature of nitrogen vapours exiting the exchanger system, in order to control the heat exchange quality between the evaporating liquid nitrogen and the circulating air.

The oxygen concentration control system 23 inside the cryogenic chamber is mounted on the side wall of the upper channel L. It is comprised of a gas detector 10, whose sensor module is located in the measuring box 12, connected to the copper pipe 11 with wound and externally insulated heating cable 6. The other end of the copper pipe is located in the lower outlet channel, wherein it should be noted that this end is bended in the direction of the channel axis in such a way that the air circulating inside the chamber spontaneously (passively) flows into the copper pipe and into the measuring box. A transverse blower 14 connected to the measuring box and fixed to its right side wall from the outside is an additional element providing an appropriate flow of the sampled air, wherein an opening was made, allowing drawing out the sampled air. The gas detector is connected to the control and alarm module 13 provided to cooperate with the gas detector and the chamber operation control system, which, in case of detecting a too low or too high concentration of oxygen in the atmosphere inside the cryochamber, will activate an alarm mode in the device allowing the patient to safely exit the chamber. The system is powered by a dedicated power supply located in the control cabinet of the device, which powers the sensor at all times when the chamber is activated.

1. Device for systemic cryotherapy with an engine room assembly comprising a cryochamber and an engine room assembly of the cryochamber, which comprises an upper inlet channel housing (8), which is connected, on the bottom, to a ventilation fitting housing (1) connected, on the bottom, to a heat exchanger housing (2), wherein, on the side wall, a cryogenic liquid supply and cryogenic liquid vapour discharge system is located, and the lower flange of the heat exchanger (2) is connected to the lower outlet channel housing (3) and to the side wall of the engine room assembly, and the device is connected to the power supply system, characterised in that the upper inlet channel housing (8) is L-shaped, and a duct fan (15) is located in the ventilation fitting housing (1), wherein a heater (9) is located between the ventilation fitting (1) and the heat exchanger (2).

2. The engine room assembly according to claim 1, characterised in that, inside the lower outlet channel housing (3), a guide vane (4) is located.

3. The engine room assembly according to claims 1 to 2, characterised in that, on the lower outlet channel exit (3a), specifically on its damper (3b), a Pt100 temperature sensor in stainless steel housing (5) is located.

4. The engine room assembly according to claims 1 to 3, characterised in that the Pt100 temperature sensor is mounted on the chamber housing using a Pt100 mounting sleeve (7), passing through the walls of the lower outlet channel housing (3).

5. The engine room assembly according to claims 1 to 4, characterised in that, on the side wall of the lower outlet channel housing (3), an oxygen concentration control system is located inside the cryogenic chamber (23), connected to the engine room assembly of the cryochamber with a copper pipe (11).

6. The engine room assembly according to claims 1 to 5, characterised in that, one end of the copper pipe (11) connecting the oxygen concentration control system (23) to the lower outlet channel housing (3) passes through the wall of the lower outlet channel housing (3), wherein this end is bended in the direction of the channel axis according the direction of airflow.

7. The engine room assembly according to claim 6, characterised in that, on the copper pipe (11), an externally insulated heating cable (6) is wound, and the other end of the copper pipe is connected to the gas detector module (10) of the oxygen concentration control system (23), which module is located in the measuring box (12).

8. The engine room assembly according to claims 6 to 7, characterised in that the measuring box (12) is connected directly to the transverse blower (14), which is fixed to the side wall of the measuring box (12) from the outside of the heat exchanger (2).

9. The engine room assembly according to claims 6 to 8, characterised in that, the measuring box (12) is connected directly to the transverse blower (14) through an opening in the side wall of the measuring box (12).

10. The engine room assembly according to claims 1 to 9, characterised in that, the oxygen concentration control system (23) is connected with a cable to a control and alarm module (23) and to a chamber operation control system located in the upper electrical box (28).
